(19) United States
(12) Patent Application Publication
(10) Pub. No.: US 2005/0244357 A1
(43) Pub. Date: Nov. 3, 2005

(54) Oil-containing emulsifiable formulations containing active agents

(76) Inventors: Eward Sieverding, St. Johann (DE); Ingo Fleute-Schlachter, Essen (DE)

Correspondence Address: FROMMER LAWRENCE & HAUG, 745 FIFTH AVENUE- 10TH FL., NEW YORK, NY, 10151, US

(21) Appl. No.: 10/968,543

(22) Filed: Oct. 19, 2004

Related U.S. Application Data
(60) Provisional application No. 60/547,872, filed on Feb. 26, 2004.

(51) Int. Cl. A61K 7/06; A61K 7/11

(52) U.S. Cl. 424/70.12

(57) ABSTRACT

The invention relates to an oil-containing emulsifiable formulation which is substantially free of water comprising a) one or more agriculturally acceptable active agents, b) one or more organic solvents, c) at least one emulsifying surfactant system that forms an oil-in-water emulsion when the formulation is added to water, d) a formulation synergist comprising one or more organo-modified siloxanes e) optionally other additives.

### RELATED APPLICATIONS

This application claims priority to provisional application U.S. Ser. No. 60/547,872, filed Feb. 26, 2004, herein incorporated by reference.

### BACKGROUND OF THE INVENTION

1. Field of the Invention

The invention relates to improved pesticidal activity of oil-containing emulsifiable formulations, such as emulsifiable concentrates (EC), that are essentially free of water and contain agriculturally acceptable active agents by adding undiluted organosilicones to the formulation, as well as to oil-containing emulsifiable formulations that comprise an agriculturally acceptable active agent (or active ingredient) and an undiluted silicon.

2. Description of the Art

Emulsifiable concentrates (EC) generally contain an active ingredient or agent, a water immiscible solvent, emulsifier and other additives such as stabilizer, sticker, antifoams, wetting agents etc. Sometimes, small amounts of water are added to form a homogeneous formulation but essentially they are water free.

EC are widely used because of their simple way of manufacturing. There have been numerous publications about this formulation type, e.g. “Surfactants in Agrochemicals”, Marcel Dekker Surfactant Science Series Vol. 54, edited by Tharwat Tadros. For application in the field, such formulations are diluted in water, forming an o/w emulsion (oil-in-water) that allows a homogeneous distribution of active ingredients (pesticides as synonym with crop protection agents or biocides) on crops. U.S. Pat. No. 6,566,308 B1 reports that the presence of solvents in EC lead to a higher degree of systemicity and higher overall activity of the pesticides compared to other solid or aqueous based formulations of pesticides.

Traditionally, organic solvents such as xylene and solvent naphtha are used as solvent in EC. These compounds show a high capability of dissolving a wide range of active ingredients because of the presence of aromatic groups. Alternative solvents are mineral and vegetable oils and their derivatives, especially esterified fatty acids and methylated seed oils (MSO).

Suitable emulsifiers are usually blended and not used as single component. Such a blend can consist of two non-ionic surfactants. A mixture of a non-ionic and an anionic surfactant is used more frequently. The ratio of the two components is critical to find the most suitable HLB for the solvent. HLB or the hydrophilic-lipophilic balance system is well known to those who are skilled in the art. This scale is based on the relative percentage of lipophilic and hydrophilic surfactants.

The activity of EC formulations can be improved by the addition of adjuvants. U.S. Ser. No. 6,566,308 B1 discloses the use of adjuvants selected from the group consisting of an alcohol alkoxylate, a water immiscible N-alkylpyrrolidone and a mixture thereof. However, no reference is made to silicone containing additives or adjuvants.

Organo-modified siloxanes (OMS) or simply silicone surfactants increase the uptake of pesticides and hence their activity as described in Novel Surfactants, Marcel Dekker Surfactant Science Series Vol. 114, edited by Krister Holmberg, pp. 585 ff, herein incorporated by reference. A prominent class of OMS are polyether siloxanes. In polyethers, oxirane derivatives e.g. ethylene oxide, propylene oxide, butylene oxide, are added to a starting alcohol. There are different orders of addition, blockwise, statistical or a mixture thereof.

Dimethoate applied alone gave good control of thrips in cotton as published in the Proceedings of the Fifth International Symposium on Adjuvants for Agrochemicals, 1998, Vol. I., pp. 342 ff, edited by Patrick McMullan. An organosilicone improved the efficacy of thrips control when tank-mixed with the active. Thrips control was less when the organosilicone was formulated in can with the active rather than applied as a tank-mix adjuvant. However, a pure organosilicone was not used. In the publication the added adjuvant was an organosilicone blended with a non-ionic surfactant.

Tank-mix is the separate addition of pesticides and/or adjuvants to the tank in a concentration that is ready to be sprayed on plants. In can formulations (means: part of the formulation), simply formulation, pre-mixing or pre-addition are all synonyms. This is the preparation of a pesticidal concentration containing additives. ECs are one example for a formulation.

### OBJECT OF THE INVENTION

An object of the present invention was, therefore, the development of improved oil-containing emulsifiable formulations, such as EC, that are essentially free of water and contain agriculturally acceptable active agents. This and other objects will become apparent from the Description of the Invention.

### DESCRIPTION OF THE INVENTION

Surprisingly, it has now been found that the inclusion of an undiluted organosilicone into oil-containing emulsifiable formulations of an agriculturally acceptable active agent enhances synergistically the pesticidal activity. Surprisingly and unexpectedly, and currently not explainable, pre-mixing of organosilicone to an oil-containing emulsifiable formulation, which is essentially free of water, such as an emulsifiable concentrate (EC), oil dispersion (OD) or suspo-emulsion (SE) formulation before adding to a spray tank enhanced the biological activity of an active agent, such as a pesticide, significantly more than adding the organosilicone and an oil-containing formulated active agent (e.g. pesticide) separately to a spray tank. Adjuvants which act in this way are described herein as formulation synergists. Enhancement of biological efficacy by direct incorporation into the formulation can be expected from all liquid formulation types which can be prepared essentially without water, such as emulsifiable concentrates (EC), suspension concentrates for direct application (SD), spot application (SA) or pour on (PO) formulations, oil dispersible powder (OP) formulations, oil miscible liquid (OL) formulations, oil miscible flowable concentrate (OF) formulations, oil dispersion (OD) or suspo-emulsion (SE) formulations; the formulation types are described by Crop Life International: Catalogue of pesticide formulation types and international coding system. Technical Monograph nr. 2, 5^{th} edition (http://www.croplife.org/library/documents/Technical%20Monographs/Technical%20Monograp h%20N%C2%B02-%20March%202002.pdf). Accessed Nov. 17, 2003.

This invention relates to emulsifiable concentrate formulations which are substantially free of water comprising
a) one or more agriculturally acceptable active agents,
b) one or more organic solvents,
c) at least one emulsifying surfactant system which forms an oil-in-water emulsion when the formulation is added to water,
d) a formulation synergist comprising one or more organo-modified siloxanes which are not blended with other surfactants (i.e., “pure siloxanes”),
e) optionally other additives.

This invention also relates to formulations wherein said organo-modified siloxanes are at least one compound of the formula (I) 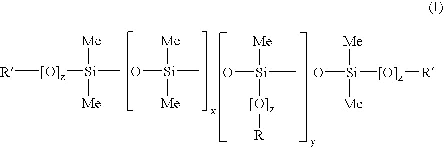
where
z=0 or 1
x=0 to 100
y≧0
R′═R or alkyl
R═CₙH₂ₙO(CₑH₂ₑO)ₚ K and/or CₘH₂ₘ₊₁
n=3 to 4,
m=1−30
e=the same or different 2 to 4,
p≧3,
K═H, an alkyl of 4 carbons or less or an acetyl group with the provisio that the molecule contains at least one moiety R.

Preferred organosiloxanes are such of formula (I) wherein
z=0
x=0
y=1−4
R′=methyl
R═CₙH₂ₙO(CₑH₂ₑO)ₚK
n=3 to 4,
e=2 to 4.

Especially preferred are organosiloxanes which are a mixture of ethylene oxide and propylene oxide in a ratio of 1:1 to 1:4, with 1:1 to 1:3 being especially preferred.

The formulation contains about 0.1% to about 50% by volume of at least one component of formula (I) especially about 2% to about 35% by weight and is substantially free of water.

The invention relates to oil-containing emulsifiable formulations, e.g., emulsifiable concentrate (EC), oil dispersion (OD), or suspo-emulsion (SE), which are substantially free or almost free of water. Such formulations can contain one or several agriculturally acceptable active ingredients.

Agriculturally acceptable active agents are those which are registered for use in individual countries to protect plants or to avoid damage from plants or to avoid yield losses of crops. Such active ingredients can be of synthetic as well as of biological nature. Such ingredients can also be extracts as well as antagonistically active organisms. They are often also commonly named pesticides which are known by people skilled in the art. Pesticides include acaricides (AC), algicides (AL), attractants (AT), repellents (RE), bactericides (BA), fungicides (FU), herbicides (HB), insecticides (IN), molluscicides (MO), nematicides (NE), rodenticides (RO), safeners (SA), sterilants (ST), synergists (SY), viricides (VI), plant growth regulators (PG), and which are used alone or in combinations thereoff. Preferred are HB, FU, IN, AC, SA, and in particular HB, FU, IN.

Chemical classes, compounds of active ingredients or organisms which are agriculturally acceptable are, together with their use or uses, are listed for example in ‘The Pesticide Manual’, 13^{th} edition, 2003, The British Crop Protection Council, in ‘The BioPesticide Manual’, 2^{nd} edition, 1998, The British Crop Protection Council, and in other there cited literature; these documents are herein incorporated by reference.

Non-limiting examples of agriculturally acceptable active ingredients include, for example, for AC: abamectin, bromocyclen, clofentezin, diazinon, tebufenpyrad; for AL exemplified by dichlorophen; for AT exemplified by 3,7-dimethyl-2,6-ocatien-1ol; for RE exemplified by anthraquinon, citronellol, daphne-oil, guazatin, sebazinic acid; for BA exemplified by streptomycin, pentylphenol, dichlorophen; for FU exemplified by epoxiconazole, fenpropidine, fenpropimorph, difenoconazole, tebuconazole, prochloraz, famoxadone, propiconazole, spiroxamine, kresoxim-methyl, pyraclostrobin, fluquinconazole, azoxystrobin, carbendazim, fenhexamid, carpropamid; for HB exemplified by trifuralin, metalachlor, quizalofop-P, pyridat, bromoxynil, phenmedipham, ethofuimesan, haloxyfop-R, fluroxypyr, metosulam, metazachlor, clomazone, fluazifop-P, bentazone, clodinafop-propargyl, dicamba, metamitron, nicosulfuron, araquat, picolinafen, pendimethalin, rimsulfuron, quinclorac; for IN exemplified by acetamiprid, cypermethrin, imidacloprid, pirimicarb, esfenvalerat, dimethoat, azadirachtin, mineral oils, pyrethrine, rape seed oil, oxydemeton-mythyl spinosad; for MO exemplified by ethanol, thiodicarb, metaldehyde, mcpa-thiethyl; for NE exemplified by benfuracarb, carbosulfan, fenamiphos, oxamyl, thionazin; for RO exemplified by papain, bromethalin, calciferol, coumafuryl, fluoroacetamid; for SA exemplified by benoxacor safener, colquintocet-mexyl safener, fenchlorim safener, mefenpyrdiethyl safener; for ST exemplified by dazomet, 1,2 dichloropropan; for SY exemplified by piperonylbutoxid, sulfaquinoxalin; for PG exemplified by gibberilic acid, indolylbutylic acid, trinexapac-ethyl.

Biological actives can be bacteria, actinomycetes, fungi, nematodes, viruses, or compounds or parts thereoff. Preferred are Bacillus spp. (z.B. B. sphaericus IN, B. subtilis FU; B. thuringiensis IN with e.g. B. thuringiensis aizawai, B. thuringiensis israelensis, B. thuringiensis kurstaki, B. thuringiensis tenebrionis); Pseudomonas spp., Streptomyces griseoviridis FU; Ampelomyces quisqualis FU; granulo-viruses IN or for example nuclear polyhedrose virus IN.

Ingredients also contemplated in this invention include those that are used in the non-crop, or non-agricultural sector to protect forestry, all kinds of seed, all kinds of industrial areas and/or materials or construction materials or objects inclusive homes and gardens against insects, diseases, un-wanted plants and the like, as well as against bacteria, viruses, animals, snails, molluscs and the like.

Active ingredients are preferably used in the formuations in a concentration of about 0.0005 to about 80% (w/w), more preferably in a concentration of about 0.05 to about 50% (w/w). The methods of preparing emulsion concentrates are known: see, for example, U.S. Pat. No. 6,566,308 B1 or U.S. Ser. No. 2003-0118614 A1, both herein incorporated by reference. It is well known within the people skilled in this art how to prepare suspo-emulsions. Formulations can also contain other additive compounds which are commonly included; non-limiting examples of these additive compounds are antifoams, anti-frost compounds, thickeners, regulators, dyes, water-free additives, non-aqueous solvents and the like. These additive compounds, as well as sources to obtain them, would be well-known to a practitioner of this art.

Non-limiting examples of solvents that may be used in such formulations of the invention include seed oils e.g. canola oil, methylated and ethylated fatty acids, xylene, aromatic hydrocarbon fluid like the Solvesso® line of solvents, hydrocarbon solvents like solvent naphtha, Shellsol® line of solvents, and the like including such grades that are naphthalene depleted, terpene alcohols, ketones such as cyclo-hexanone.

The other components are used in the same concentrations as known in the state of the art.

Formulations that are substantially free of water include, for example, those that contain less than 5% water, more preferably, those that contain less than 3% water.

This invention is exemplified from the following non-limiting examples.

### EXAMPLE

Additive 1 is a preferred organosiloxane of formula (I), also designated as trisiloxane surfactant, and is available from Goldschmidt AG, Germany under the trade name BREAK-THRU® S 240.

Additive 2 is a comb-like siloxane surfactant available from Goldschmidt AG, Germany under the trade name BREAK-THRU® OE 441.

Preparation of Example Formulation A:

200 ml of Additive 1 (Break-Thru® S 240 of Goldschmidt AG) were directly mixed with 400 ml of the commercially available herbicide Topik® (registered product of Syngenta). Topik is an EC formulation which contains 80 g/l clodinafop-propargyl and 20 g/l of the safener cloquintocet-mexyl. Topik® EC was used as a model EC. The example formulation A was shaken and turned out to be stable after submission to an accelerated storage test at 54° C. for 14 days. Such accelerated storage tests are described in the CIPAC Handbook Volume F, Method MT 46, and are commonly used to predict a two years stability of formulations.

Preparation of Example Formulations B and C:

2 ml (for formulation B) or 5 ml (for formulation C) of Additive 2 (Break-Thru® OE 441 of Goldschmidt AG, Essen) were directly mixed with 98 ml or 95 ml of the commercially available herbicide Topik® (registered product of Syngenta). Topik is in these studies a model EC of a pesticide. Topik® EC contains 80 g/l clodinafop-propargyl and 20 g/l of the safener cloquintocet-mexyl. The mixtures (example formulations B and C) were shaken, within a bottle, by hand. The mixtures were stable after submission to an accelerated storage test at 54° C. 14 days according to CIPAC method MT 46.

Preparation of Example Formulations D, E and F:

The following mixtures with pesticides were also formulated to test their stability and the suitability of preparation:

### Example D

Vertimec® EC 18 is available from Syngenta/Germany and contains the insecticide abamectin as active ingredient:
D 1 Vertimec® 9 ml with 2 ml BREAK-THRU® S 240
D 2 Vertimec® 9 ml with 0.5 ml BREAK-THRU® OE 441
D 3 Vertimec® 9 ml with 0.5 ml BREAK-THRU® OE 444

### Example E

Betanal® Expert EC is a herbicide available from Bayer/Germany and contains 25 g/l desmedipham, 151 g/l ethofumesate and 75 g/l phenmedipham as active ingredients:
E 1 Betanal® 17.5 ml with 2 ml BREAK-THRU® S 240
E 2 Betanal® 17.5 ml with 0.85 ml BREAK-THRU® OE 441
E 3 Betanal® 17.5 ml with 0.85 ml BREAK-THRU® OE 444

### Example F

Targa® Super EC is a herbicide from Bayer/Germany and contains the 46.3 g/l quizalofop-P as active ingredients:
F 1 TargaS Super 20 ml with 2 ml BREAK-THRU® S 240

F 2 Targag Super 20 ml with 1 ml BREAK-THRU® OE 441

TABLE
Stability of example EC formulations with additives

| | Pesticide | | | | | |
|---|---|---|---|---|---|---|
| | BREAK-THRU ® | | BREAK-THRU ® | | BREAK-THRU ® | |
| | S 240 | | OE 441 | | OE 444 | |
| | Storage Temp. | | | | | |
| | 22° C. | 54° C. | 22° C. | 54° C. | 22° C. | 54° C. |
| Vertimec ® | D1: clear | D1: clear | D2: clear | D2: clear | D3: turbid | D3: Phase |
| | | | | | | separation |
| Betanal ® | E1: clear | E1: clear | E2: clear | E2: clear | E3: turbid | E3: Phase |
| | | | | | | separation |
| Targa ® | F1: clear | F1: clear | F2: clear | F2: clear | — | — |

It resulted that all EC formulated pesticides were stable and pre-mixable with Break-Thru S 240 and Break-Thru OE 441; the pre-mixability of Break-Thru OE 444 may depend on the solvent in the formulation.

Biological Efficacy of Formulation A:

A field trial was laid down in wheat in Germany. The wheat field was heavily infested with black grass (Alopecurus myosuroides). The objective of the trial was to compare the efficacy of Additive 1 either incorporated into formulation A or tank mixed with an EC formulated crop protection agent. The commercial herbicide Topik® EC (80 g/l clodinafop-propargyl and 20 g/l of the safener cloquintocet-mexyl) was used as a model EC product. In the case of formulation A, the additive 1 was mixed with the herbicide before adding to water in a spray tank, and in case of tank mixing, the additive 1 and the herbicide product were added separately to water of the spray tank.

In the case of formulation A, 600 ml per ha were added to the spray tank and in the case of the tank mixture 400 ml Topik® EC per ha and 200 ml Break-Thru® S 240 per ha were separately added to the spray tank before spraying. The equivalents of 300 1 per hectare spray solution were sprayed to 15 m² field plots using Agrotop AirMix 110 04 nozzles and 1.8 bar pressure. There were four randomly distributed field plots per treatment in the trial. The trial had also untreated (none-sprayed) plots, and plots which were sprayed with the equivalent of 400 ml of Topik® EC alone per ha diluted in 300 1 water per ha. Spraying was carried out when the crop and the grass weed where at crop growth stage 14 (BBCH scale). The effects of the treatments, thus their efficacies, were evaluated 6 weeks after treatment by assessing the percent weed control in the treated plots as compared to the weed cover in the untreated plots of each replicate; such assessment methods are well known by people skilled in the art. The grass weed ground coverage was 58% in the untreated controls at the time of assessment. The average percent weed control of the 4 field replicates was calculated and was found to be:

| | |
|---|---|
| Topik ® EC at 400 ml/ha: | 39% weed control |
| Topik ® EC at 400 ml/ha plus 200 ml/ha Additive 1 | 93% weed control |
| within formulation A (pre-mixed): | |
| Topik ® EC at 400 ml/ha plus 200 ml/ha Additive 1 | 86% weed control |
| tank mixed: | |

The statistical analyses of the values (Analysis of Variance, comparison of means with Tukey Test, using the Statistix for Windows 2.0 programme from Analytical Software, Tallahassee, Fla., USA) revealed that Additive 1 increased significantly (p=0.01) the weed control compared to Topik® EC alone. Surprisingly, and unexpectedly, the premixing of Additive 1 with the EC of Topik® increased significantly (p=0.05) the weed control more than the tank mixing of the two products. Adjuvants with such effects as Additive 1 (and more broadly those in the sense of formula I of this patent) are herewith named: formulation synergists. A formulation synergists is herewith described as a compound as part of or within a formulation which creates a greater biological effect of the active ingredient when part of the formulation than when added separately to the active ingredient in a tank mixture. Such naming follows the description of synergistic effects by Colby, S. R., 1967: “Calculating Syngergistic and Antagonistic Responses of Herbicide Combinations”, Weeds, 15: S. 20-22, which defines combinations as being synergistic when the combination of two compounds has a higher efficacy than that what can be expected from the combination. Indeed, this definition is here also applicable: The to be expected efficacy would be the combination of Additive A+Herbicide in tank mix; all additional effect is synergistic. The results of the above experiment may be an example for the improvement of the biological activity of EC's in general. Such improvements have practical consequences: They can lead either to improved efficacy of a plant protection agent which is formulated as an EC, or it can result in lower active ingredient amounts per ha needed to obtain excellent and agronomic acceptable efficacy.

Biological Efficacy of Formulations B and C

A field trial was laid down in wheat in Germany. The wheat field was heavily infested with black grass (Alopecurus myosuroides). The objective of the trial was to investigate the effectiveness of Additive 2 to improve the efficacy of an EC formulated crop protection agent. The commercial herbicide Topik® EC which contains 80 g/l clodinafop-propargyl as the active herbicidal ingredient and 20 g/l of the safener cloquintocet-mexyl was used as a model EC product. Additive 2 had been incorporated into Formulation B at 2% v/v, and in Formulation C at 5% v/v, and these two formulations were compared with the efficacy of Topik® EC. Formulations B, C and Topik® EC were applied at a dose of 24 g active ingredient (a.i.) per ha of clodinafop-propargyl. Equivalent volumes of the formulation were added to water (300 l/ha of spray volume) and equivalent amounts of spray solutions were sprayed to 15 m² field plots using Agrotop AirMix 110 04 nozzles and 1.8 bar pressure. There were four randomly distributed field plots per treatment in the trial. The trial had also untreated (none-sprayed) plots. Spraying was carried out when the crop and the grass weed were in crop growth stage 14 (BBCH scale). The effects of the treatments, thus the efficacies of the formulations, were assessed 6 weeks after treatment by assessing the percent weed control in the treated plots as compared to the weed coverage in the untreated plots of each replicate; such assessment methods are well known by people skilled in the art. The grass weed ground coverage was 58% in the untreated controls at the time of assessment. The average percent weed control of the 4 field replicates was calculated and was found to be:

| | |
|---|---|
| Topik ® EC at 24 g/ha a.i. of clodinafop-propargyl: | 13% |
| Formulation B (with 2% Break-Thru ® OE 441) at 24 g a.i. | 33% |
| clodinafop-propargyl: | |
| Formulation C (with 5% Break-Thru ® OE 441) at 24 g a.i. | 48% |
| clodinafop-propargyl: | |

The statistical analyses of the values (Analysis of Variance, comparison of means with Tukey Test, Statistix programme as above) revealed that Topik® EC was significantly less active than Formulation C (p=0.05). The activity of Formulation B was not significantly different from Topik EC nor from Formulation C.

The results show that the addition of small volume percentages of Additive 2 to an EC formulation increases significantly its activity. This can lead to improved efficacy of a plant protection agent which is formulated as an EC, or it can result in lower active ingredient amounts needed per ha to obtain acceptable efficacy of a crop protection agent.

The data shown here with EC formulated herbicides are examples for the activity improvements of all EC formulated crop protection agents through the incorporation of the herein described additives of formula (I). The active ingredient may, in such formulations, be present as a single active biocide or a mixture of various active ingredients in concentrations which are often dictated by the EC formulation process. The active ingredient can be an algicide, attractant, repellent, bactericide, fungicide, herbicide, insecticide, miticide, moluscicide, nematicide, rodenticide, a crop safener, a sterilant, synergist, a viridicide, or a plant growth regulator, being present one of the above alone or in any mixture thereof.

The above description of the invention is intended to be illustrative and not limiting. Various changes or modifications in the embodiments described herein may occur to those skilled in the art. These changes can be made without departing from the scope or spirit of the invention.

1. An oil-containing emulsifiable formulation which is substantially free of water comprising:
a) one or more agriculturally acceptable active agents,
b) one or more organic solvents,
c) at least one emulsifying surfactant system that forms an oil-in-water emulsion when the formulation is added to water,
d) a formulation synergist comprising one or more organo-modified siloxane,
e) optionally other additive compounds.

2. The formulation according to claim 1 wherein said organo-modified siloxane is a compound of the formula 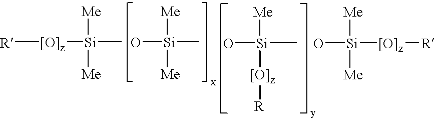
where
z=0 or 1
x=0 to 100
y≧0
R′═R or alkyl
R═CₙH₂ₙO(CₑH₂ₑO)ₚK and/or CₘH₂ₘ₊₁
n=3 to 4,
m=1−30
e=the same or different 2 to 4,
p≧3,
K═H, an alkyl of 4 carbons or less or an acetyl group with the provisio that the molecule contains at least one moiety R.

3. The formulation according to claim 2 where the organo-modified siloxane is a compound of the formula
z=0
x=0
y=1−4
R′═methyl
R═CₙH₂ₙO(CₑH₂ₑO)ₚK
n=3 to 4,
e=2 to 4.
with the provisio that the molecule contains at least one moiety R.

4. The formulation according to claim 2, wherein the oilcontaining emulsifiable formulation is an emulsion concentrate.

5. The formulation according to claim 2, wherein the oil-containing emulsifiable formulation is an oil dispersion of a suspo-emulsion.

6. The formulation according to claim 4, wherein the organic solvent is a seed oil, xylene, methylated and ethylated fatty acids, xylene, solvent naphtha, terpene alcohols, aromatic hydrocarbons, and cyclohexane.

7. The formulation according to claim 4, wherein the agriculturally acceptable active compound is a herbicide, a safener or a combination thereof.

8. The formulation according to claim 7, wherein the herbicide or safener is clodinafop-proparglyl, cloquintocet-mexyl; desmedipham, ethoftimesate, phenmedipham, quizalofop-P ethylester.

9. The formulation according to claim 4, wherein the agriculturally acceptable active compound is an acaricide.

10. The formulation according to claim 4, wherein the acaricide is abamectin.

11. A method for increasing the pesticidal activity of an agriculturally acceptable active agent formulated in an oil in water formulation which comprises first mixing an oil-containing emulsifiable formulation, which is substantially free of water and contains the agriculturally acceptable active agent, with at least one organosilicone and subsequently adding water to the mixture to form the oil-containing emulsion.

12. The method according to claim 11, wherein the oil-containing emulsifiable formulation is an emulsifiable concentrate comprising
a) one or more agriculturally acceptable active agents,
b) one or more organic solvents,
c) at least one emulsifying surfactant system that forms an oil-in-water emulsion when the formulation is added to water,
d) one or more organo-modified siloxane
e) optionally other additives.

13. The method according to claim 12, wherein the organo-modified siloxane is a compound of the formula 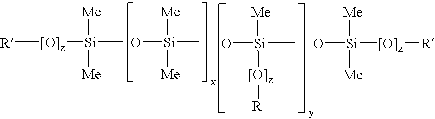
where
z=0 or 1
x=0 to 100
y≧0
R′═R or alkyl
R═CHₙH₂ₙO(CₑH₂ₑO)ₚK and/or CₘH₂ₘ₊₁
n=3 to 4,
m=1−30
e=the same or different 2 to 4,
p≧3,
K═H, an alkyl of 4 carbons or less or an acetyl group with the provisio that the molecule contains at least one moiety R.

14. The method according to claim 13, wherein the organo-modified siloxane is
z=0
x=0
y=1−4
R′=methyl
R═CₙH₂ₙO(CₑH₂ₑO)ₚK
n=3 to 4,
e=2 to 4.
with the provisio that the molecule contains at least one moiety R.

15. The method according to claim 13, wherein the agriculturally acceptable active compound is a herbicide, a safener or a combination thereof.

16. The method according to claim 15, wherein the herebicide or safener is clodinafop-proparglyl, cloquintocet-mexyl; desmedipham, ethofumesate, phenmediphan, quizalofop-P ethylester.

17. The method according to claim 13, wherein the agriculturally acceptable active compound is an acaricide.

18. The method according to claim 17, wherein the acaricide is abamectin.

19. The method according to claim 11, wherein the oil-containing emulsifiable formulation is an oil dispersion or a suspo-emulsion.
